(19) United States
(12) Patent Application Publication
(10) Pub. No.: US 2020/0330611 A1
(43) Pub. Date: Oct. 22, 2020

(54) METHODOLOGY FOR IDENTIFYING GENE DELIVERY VECTORS WITH RETINAL CELL SPECIFICITY IN NON-HUMAN PRIMATE

(71) Applicants: University of Florida Research Foundation, Incorporated, Gainesville, FL (US); The UAB Research Foundation, Birmingham, AL (US)

(72) Inventors: Shannon E. Boye, Gainesville, FL (US); Sanford L. Boye, Gainesville, FL (US); Paul D. Gamlin, Birmingham, AL (US)

(73) Assignees: University of Florida Research Foundation, Incorporated, Gainesville, FL (US); The UAB Research Foundation, Birmingham, AL (US)

(21) Appl. No.: 15/998,618

(22) PCT Filed: Feb. 16, 2017
(86) PCT No.: PCT/US2017/018240

§ 371 Date: Aug. 16, 2018

Related U.S. Application Data
(60) Provisional application No. 62/296,056, filed on Feb. 16, 2016.

(51) Int. Cl. A61K 49/00; C12N 5/079; G01N 1/28; C12N 15/86; A61K 48/00; G01N 21/64

(52) U.S. Cl. CPC A61K 49/0017; C12N 5/0621; G01N 1/28; C12N 15/86; G01N 2021/6441; A61K 48/0008; G01N 21/6428; C12N 2750/14143; C12N 2830/008; A61K 48/0075

(57) ABSTRACT

The present application provides methods of producing sortable sub-populations of retinal cells. Methods can be used in vivo. Methods can be used to produce sortable sub- populations of cells in non-human primate retina. Methods can be used in conjunction with vivo testing of gene therapy agents and ex-vivo cell sorting and analysis to determine the specificity and/or effectiveness of transgene transduction, transcription, expression, and/or function of the transgene product.

### RELATED APPLICATIONS

This application claims the benefit of U.S. provisional application No. 62/296,056, filed on Feb. 16, 2016, the entire disclosure of which is incorporated by reference herein.

### FEDERALLY SPONSORED RESEARCH

This invention was made with government support under grant number EY024280 awarded by the National Institutes of Health. The government has certain rights in the invention.

### BACKGROUND

While transgenic mice are invaluable for many studies, their utility as a model for human disease is limited by substantial differences in their ocular anatomy or retinal topography. These differences include, but are not limited to, the absence of a cone exclusive fovea (the region of the primate retina responsible for high acuity vision), differential patterns of retinal vascular and basement membrane thicknesses, and a significantly smaller globe. The differences present a challenge for translating findings in mice to human patients with retinal disease. Compared to the ocular anatomy of mice, that of non-human primates (e.g., macaque) is much more similar to that of humans.

Mouse models have been developed to evaluate retinal gene expression, but there are currently no good non-human primate models for studying the specificity and/or efficacy of expression from experimental transgene constructs in primate retina.

### SUMMARY

This disclosure provides a non-human primate model or platform for the evaluation of gene or protein expression in various ocular cells, for example different types of retinal cells.

This disclosure is based, at least in part, on experiments done to establish a macaque model and methods by which macaque retinas generated with sortable cell populations, e.g., retinal ganglion cells (RGCs) and photoreceptors (PRs). Methods disclosed herein enable the evaluation of vector tropism and/or cellular activity of gene delivery vectors and response elements (e.g., promoters).

In some embodiments, one or more different types of a non-human primate eye are differentially labeled, and a reagent (e.g., a test vector) is administered to the eye to determine whether the reagent preferentially targets and/or is preferentially active in one or more cell types relative to other cell types in the eye.

In some aspects, provided herein is a method of preparing a non-human primate retina comprising one or more sortable retinal cell populations. In some embodiments, the method comprises labeling a first retinal cell type in the non-human primate retina with a first label. In some embodiments, the method comprises labeling a second retinal cell type in the non-human primate retina with a second label (e.g., different from the first label). In some embodiments, the method comprises labeling additional cell types (e.g., 3, 4, 5, 6 or more different cells types) in the non-human primate retina with additional labels (e.g., with 3, 4, 5, 6, or more different labels).

In some embodiments, the one or more (e.g., 2, 3, 4, 5, 6, or more) sortable retinal cell populations is selected from the group consisting of: photoreceptors (PR), retinal ganglion cells (RGC), bipolar cells, retinal pigment epithelium (RPE), astrocytes, horizontal cells, amacrine cells, microglia, and Muller glia.

In some embodiments, one or more retinal cell populations is rendered sortable by labeling with a dye (e.g., delivered by injection or other suitable method of administration). In some embodiments, one or more retinal cell populations is labeled using a gene expression product (e.g., delivered using a viral vector or other suitable method of administration). In some embodiments, one or more additional cell populations is differentially labeled using one or more additional dyes and/or gene expression products. For example, any one of the methods disclosed herein can further comprise labeling a second retinal cell type with a second label. In some embodiments, a method disclosed herein comprises labeling a first, second, third, fourth, fifth, and/or sixth retinal cell type with a first, second, third, fourth, fifth and/or sixth label, respectively.

In some embodiments, one or more sortable retinal cell populations are sorted using Fluorescence-activated cell sorting (FACS). However, other known techniques to sort cell populations (e.g., based on optically discernable labels) can be used.

In some embodiments, one or more sortable retinal cell populations are labeled in vivo, In some embodiments, confirmation of differential in vivo labeling of different retinal cell types is confirmed in vivo. In some embodiments, confirmation of cell labeling is performed in-life or while the animal is living. In some embodiments of any of the methods disclosed herein, in vivo confirmation is performed using scanning laser ophthalmoscopy (SLO). In some embodiments of any of the methods disclosed herein, in vivo confirmation is performed using fundoscopy.

In some embodiments, one or more recombinant vectors are used to transduce the one or more sortable retinal cell populations to express a label (e.g., a vector carrying a gene that encodes a fluorescent protein or protein fragment). In some embodiments, a vector used to transduce the one or more sortable retinal cell population is a viral vector, e.g., an AAV vector or HSV vector. In some embodiments, a vector is an adeno associated virus (AAV). A recombinant AAV particle of any serotype may be used (e.g., AAV5, AAV7, AAV8, AAV9, or other serotype). An AAV serotype or hybrid serotype that transduces non-human primate cells, and/or retinal cells may be used in any one of the methods disclosed herein.

In some embodiments, one or more cell-type-specific promoters are utilized to drive expression of one or more transgenes in target retinal cell populations. In some embodiments, one or more cell-type-specific promoters are selected from the group consisting of: human rhodopsin kinase promoter (hGRK1), a Pleiades Mini-promoter (Ple155), a glial fibrillary acidic protein (GFAP) promoter, a red opsin promoter, a chimeric IRBPe-GNAT2 promoter, an IRBP promoter, a Grm6-SV40 enhancer/promoter, Thy1, VMD2 and Bestrophin promoter. In some embodiments, a Pleiades Mini-promoter is Ple155. In some embodiments, a red opsin promoter is PR2.1.

In some embodiments of any one of the methods disclosed herein, one or more retinal cell populations is labeled by injection of a dye. In some embodiments, an injection is administered to the lateral geniculate nucleus. In some embodiments, an injection is administered to the lateral geniculate nucleus, and the dye is TRITC/Biotin-labeled Dextran (MICRO-RUBYTM). In some embodiments, an injection is administered intravitreally.

In some embodiments of any one of the methods disclosed herein, an injected dye is selected from the group consisting of: Brilliant Blue G, Mitotracker Orange, Mitotracker Green, Celltracker Orange, Celltracker Green, and monochlorobimane.

In some embodiments, a labeling agent is administered by intravitreal injection or by sub-inner-limit-membrane injection (subILM).

In some embodiments, a labeling agent is introduced by bathing a portion of tissue in the labeling agent which then moves in a retrograde manner to label specific cells or tissues.

In some embodiments, methods disclosed herein are useful for testing the activity of new regulatory elements (e.g., a promoter) for which cell-specificity or activity is not known. Accordingly, some embodiments of methods disclosed herein further comprise administering one or more vectors containing one or more test promoters with a reporter gene to the retina in vivo. In some embodiments, the activity of the one or more test promoter can then be evaluated in different retina cells after sorting.

In some embodiments, methods disclosed herein are useful for testing the activity of new gene delivery vectors (e.g., rAAV particles) for which cell-specificity or activity is not known. Some embodiments further comprise administering one or more functional gene therapy vectors to the retina in vivo. Some embodiments further comprise administering one or more test vectors to the retina in vivo. A test vector can be administered to a non-human primate either before or after different retinal cells are labeled. In some embodiments, one or more test vectors are administered to a non-human primate eye after one type of cell is labeled but before another type of cell type is labeled. In some embodiments, one or more test vectors are administered at the same time as one or more retinal cells types are being labeled. In some embodiments, the targeting and/or activity of the one or more test vectors can then be evaluated in different cell types after sorting.

Accordingly, some embodiments further comprise determining an expression level or activity level of one or more labels, transgenes, promoters, or other agents in one or more sortable cell types. In some embodiments, the expression level or activity level is compared to a reference level.

Some embodiments further comprise determining whether one or more labels, transgenes, promoters, or other agents are more highly expressed or more active in a first retinal cell type relative to a second retinal cell type.

These and other aspects are described in more detail herein and illustrated by the non- limiting examples and drawings.

### BRIEF DESCRIPTION OF DRAWINGS

FIG. 1 illustrates a non-limiting embodiment of a method for labeling retinal cells ex vivo;

FIG. 2A illustrates a first non-limiting example of retinal cells sorted based on differential labeling of different retinal cell types;

FIG. 2B shows different gene expression profiles in retinal cell populations sorted as illustrated in FIG. 2A;

FIG. 3A illustrates a second non-limiting example of retinal cells sorted based on differential labeling of different retinal cell types;

FIG. 3B shows different gene expression profiles in retinal cell populations sorted as illustrated in FIG. 3A;

FIG. 4 illustrates a non-limiting embodiment of a method for labeling retinal cells in vivo;

FIG. 5 shows a non-limiting example of differentially labeled photoreceptors and retinal ganglion cells using a method illustrated in FIG. 4;

FIG. 6 shows a non-limiting example of different gene expression profiles in differentially labeled and sorted retinal cells, FIG. 6A illustrates different retinal regions (superior retina, fovea, and inferior retina) from which labeled cells were obtained, FIG. 6B shows cell sorting (using FACS) of the differentially labeled cells from the superior retina, fovea, and inferior retina, in the left, center, and right panels respectively, and FIG. 6C shows different gene expression profiles of the different retinal cell populations sorted as illustrated in FIG. 6B for the superior retina, fovea, and inferior retina, in the left, center, and right panels respectively; and,

FIG. 7 illustrates a non-limiting example of M opsin and S opsin expression in fovea, superior retinal cells, and inferior retinal cells that were sorted based on differential labeling.

### DETAILED DESCRIPTION

Of the commonly used animal models, non-human primates such as macaques (genus Macaca) have ocular characteristics that are most similar to that of the human eye. Therefore, the ability to generate non-human primate (e.g., macaque) retinas with sortable cell populations is very beneficial to both basic and translational studies of the primate (including human) retina. Methods and compositions provided herein allow for such studies, which allow the identification of novel reagents designed to target specific cells of the retina, e.g., retinal ganglion cells (RGCs) and/or photoreceptors (PRs) in a species that is phylogenetically and anatomically similar to human. These methods do not incorporate steps such as fixation that are incompatible with the recovery of nucleic acids. Methods and compositions provided herein enable the evaluation of vector tropism and/or cellular activity of certain response elements (e.g., promoters), e.g., by screening of intravitreally-delivered gene therapy vectors in a clinically relevant species.

Methods and compositions provided herein relate to experiments that show that non-human primate (e.g., macaque) PRs and RGCs can be simultaneously labeled in-life and thereafter enriched and isolated by methods such as FACS (fluorescence-activated cell sorting).

Accordingly, aspects of the application relate to methods and compositions for selectively labeling and/or sorting different retinal cell types. Aspects of the application also relate to using retinal cell sorting methods to evaluate the cell specificity and/or effectiveness of different agents that may be used for targeting the eye of a subject (e.g., to treat one or more ocular conditions and/or to deliver one or more molecules to the eye of a subject, for example of a mammalian subject, e.g., a human subject).

In some embodiments, one or more different retinal cell types are labeled ex vivo (e.g., in an explanted or emplanted eye). In some embodiments, one or more retinal cell types are labeled in-life or in situ (e.g., in vivo, for example in a mammalian eye (e.g., a non-human primate eye (e.g., a macaque eye)). In some embodiments, labeled retinal cells are isolated from a retina and sorted based on the presence or absence of a label. In some embodiments, different retinal cell types are labeled with different labels, and the differentially-labeled retinal cell types are separated from each other based on the presence of the different labels. In some embodiments, cells with different labels are separated using a cell sorter (e.g., a fluorescence-activated cell sorter, or other suitable cell sorter). For example, a labeled retina can be obtained from an eye and the retinal cells can be dissociated using any suitable technique prior to separating the cells based on the presence of one or more different labels.

In some embodiments, a reagent is administered to an eye (e.g., to a retina) in addition to labeling one or more retinal cell types. In some embodiments, a retina is labeled and the labeled retina is contacted with a reagent (e.g., a test article, a vector, a gene construct, a small organic or inorganic molecule, or a plurality thereof). In some embodiments, a retina is contacted with a reagent before the retina is labeled. In either case, the labeled retinal cells that have been contacted with the reagent can be separated as described herein and the presence, amount, and/or activity of the reagent in one or more different retinal cell types can be determined. This method can be used to evaluate the retinal cell-type specificity and/or delivery effectiveness of one or more reagents that are contacted to the eye (e.g., retina) of a subject. In some embodiments, reagents are contacted to the eye in vivo. In some embodiments, reagents are contacted to the eye in an in vitro setting.

In some embodiments, two or more (e.g., 2, 3, 4, 5, 6, 7, 8, 9, 10, or more) different retinal cell types are labeled with different labels. In some embodiments, specific retinal cell types can be targeted by directly delivering a label to target retinal cells, their axonal projections (or to one or more anatomical regions of a brain from where the label is transported to target retinal cell bodies, e.g., via retrograde transport). For example, label can be injected into the lateral geniculate nuclei (LGN) to retrogradely label retinal ganglion cells (RGCs). In some embodiments, RGCs are labeled by injecting a label into the LGN and PRs are labeled by administrating a different label into the subretinal or intravitreal space. Accordingly, in some embodiments provided herein is a combinatorial approach to create, in-life, non-human primate (e.g., macaque) retinas containing both sortable PRs and RGCs, and methods for efficiently isolating and/or validating each respective cell population. In some embodiments, the brain is stereotaxically blocked in the coronal plane at the level of the brainstem and sectioned for imaging. In some embodiments, sections through the LGN are of particular interest.

In some embodiments, specific retinal cell types can be targeted using a delivery vehicle or vector that targets certain cells types (e.g., to label those cells with a detectable label). For example, recombinant AAV (rAAV, for example rAAV of serotype 5—rAAV5) particles can be used to target photoreceptor cells (e.g., by delivering the rAAV particles into the eye, for example via subretinal injection, intravitreal injection, intravascular injection, or other delivery route to the eye). In some embodiments, the rAAV particles can be labeled. In some embodiments, the rAAV particles comprise a recombinant genome encompassing a transgene that encodes a detectable label (e.g., a luminescent or fluorescent protein, for example Green Fluorescent Protein (GFP) or other protein that is detectable or that produces a detectable signal). In some embodiments, rAAV particles with different cell-specific promoters can be used to further target one or more subsets of photoreceptor cells (e.g., to differentially label one or more rod or cone cell types by expressing one or more different detectable labels in different photoreceptor cell types). In some embodiments, other virus particles with different cell specificities can be used to target different retinal cell types. In some embodiments, a combination of different techniques (e.g., selected from targeted injection, cell-type specific vectors, cell-type specific promoters, etc., or any combination of two or more thereof) can be used to differentially label two or more different retinal cell types.

In some embodiments, methods of differentially labeling and sorting retinal cell types can be used to evaluate the cell-type specificity and/or the effectiveness of delivery of one or more reagents that are provided to the retina (e.g., via subretinal injection, intravitreal injection, intravascular injection, other form of injection, topical delivery, or other suitable delivery route). In some embodiments, a reagent can be a small molecule, a recombinant gene, a viral vector (e.g., including a recombinant gene) or other reagent. In some embodiments, a reagent can include an aptamer or library of aptamers. In some embodiments, a reagent can include a recombinant virus or a library of different recombinant viral particles. In some embodiments, a reagent includes a promoter and a reporter gene. In some embodiments, a library of different promoter constructs can be used. Methods described herein can be used to evaluate the different reagents and/or select reagents (e.g., from a library) that have desired targeting properties (e.g., cell-specific delivery and/or expression) and/or delivery effectiveness (e.g., expression level). For example, retinal cells can first be differentially labeled. Subsequently, or coincidentally, a library of test reagents (e.g., a library of different vectors, promoters, aptamers, or other reagents) is administered to a retina (e.g., subretinally, intravitreally, or via any other suitable route). A library of test reagents might alternatively or also be administered to a retina before differential labeling of retinal cells. Thereafter, cells are isolated and sorted to identify different cell types. Different cells types are evaluated for the presence or activity of different test reagents (e.g., for transcripts of different test genes in a library). In some embodiments, statistical analysis is performed to evaluate which member of a library is most robustly transduced or active in different cell types. In some embodiments, a control eye is used as a reference for such analysis. In some embodiments, a control eye is the other eye of the same animal.

In some embodiments, different retinal cell types that can be targeted include, but are not limited to, Muller glia (e.g., labeled via vital dyes), astrocytes (e.g., labeled via intravascular injection of sulforhodamine dyes as shown by Appaix et al., PLoS One. 2012; 7(4):e35169), and cone photoreceptors (e.g., labeled via rAAV5 vector expressing fluorescent protein under the control of a cone specific promoter). In some embodiments, different labels (e.g., different dyes and or luminescent proteins) can be used for different cell types.

Accordingly, in some embodiments the present application provides methods of rendering sub-populations of retinal cells sortable for use in the development of ocular therapies (including for example gene therapies that target one or more retinal cell types).

In some embodiments, methods described herein can be used to assist in the development of targeted therapies or diagnoses for one or more of the following non-limiting examples of diseases or conditions: age-related macular degeneration, retinitis pigmentosa, Leber congenital amaurosis, inherited juvenile macular degeneration including but not limited to Stargardt's disease, choroideremia, achromatopsia, X-linked retinoschisis, glycogen storage disease, Usher syndrome, Bardet-Biedl syndrome, cone and rod dystrophies, Batten disease, congenital stationary night blindness (CSNB), optogenetic approaches restoring visual function, glaucoma (e.g., protection of retinal ganglion cells), diabetic retinopathy, and Lebers hereditary optic neuropathy (LHON).

In some embodiments, mammalian retinal cells are assayed as described herein. In some embodiments, the mammal is a non-human primate. In some embodiments, retinal analysis using retina from one or more other animal models may be used according to methods described in this application, including for example one of the following non-limiting models: canine models of achromatopsia, LCA, Retinitis pigmentosa (recessive and dominant), congenital stationary nightblindess, glaucoma and Stargardt disease, feline models of LCA, sheep models of achromatopsia, and transgenic pig models of cone rod dystrophy (CORD6) and dominant RP.

Clinical testing has demonstrated the potential of gene therapies to impact retinal diseases. For example, clinical trials for RPE65-Leber congenital amaurosis have shown the ability to transduce retinal pigment epithelium for corrective gene therapy. Various retinal diseases originate in pathologies and/or functional shifts in a range of different cell types. However, it remains a challenge to identify therapeutics (e.g., gene therapy treatments) that efficiently deliver appropriate therapeutic molecules to targeted cell types while minimizing off-target effects.

Transgenic rodent lines that constitutively express reporter genes in specific cell types can be generated in order to facilitate the study of the localization of gene therapy vectors, the localization of functions of gene therapy expression system, and the localization of the effective delivery of gene therapy payloads into specific cells or tissues, for example, by examining the collocation of the reporter gene and the gene therapy agent. For example, transgenic mouse models with constitutively fluorescent retinal cells have been used to quantify cell-specific transduction by Adeno Associated Virus (AAV). For example, a method for quantifying relative transduction of photoreceptors by recombinant Adeno Associated Virus (rAAV) vectors in Rho-GFP mice has been used to identify a rationally designed capsid variant, AAV2(quadY-F+T-V), capable of outer retinal transduction following intravitreal injection (Kay et al., PLoS One. 2013, 8(4):e62097). However, rodent models have significant limitations in their parallel to human retinas. For example, clinical translatability (e.g., to humans) can be limited by significant differences between rodent and primate eyes. In this regard, non-human primate (NHP) faithfully recapitulates the anatomy of the human eye. The prohibitive cost and time required to generate an NHP model by germline transgenesis, however, highlights the usefulness of the approach described in this application.

In some embodiments, techniques described in this application are useful for the development and evaluation of therapeutic compositions (e.g., viral vectors for gene therapy) that effectively target human retinal cells (e.g., by assaying non-human primate retinal cells). Given their involvement in most inherited retinal diseases (IRDs), photoreceptor (PR) cells are targeted in some embodiments. In some embodiments, retinal ganglion cells (RGCs) are targeted due to their role in glaucoma pathology and as an emerging target for optogenetic mediated vision restoration. Bipolar cells also can be targeted, for example, in view of their involvement in retinal disease and as an emerging target for optogenetics. Further retinal cell targets include Muller glia, astrocytes and microglia, given their involvement in neuroprotection, ionic homeostasis, and macrophagic function, respectively. Therapeutic targeting of one or more of these (e.g., 1, 2, 3, 4, 5, 6, 7, 8, 9, 10, or more) and/or other retinal cell types can be evaluated using retinal cell labeling and sorting methods described in this application.

In some embodiments, the present application describes a combinatorial approach for creating NHP retinas with sortable retinal cell populations that does not require germline transgenesis. This avoids the problem of creating transgenic primates, while facilitating the localization and evaluation of gene therapies in the retinas of non-human primates, providing a powerful translational vehicle to design and test retinally-directed gene therapies of clinical relevance to human subjects.

In some embodiments, the present application provides methods of preparing a non-human primate retina with retinal cell populations that can be sorted without requiring germline transgenesis of the non-human primate.

In some embodiments, methods can be used to sort retinal cell populations that include, but are not necessarily limited to, photoreceptors (PR), retinal ganglion cells (RGC), biopolar cells, retinal pigment epithelium (RPE), astrocytes, microglia, or Muller glia. In some embodiments, such cell populations are rendered capable of being sorted by labeling one or more of the cell populations with a dye, a detectable RNA, a detectable protein, or other label, or any combination of two or more thereof (e.g., different labels for different retinal cell types). In some embodiments, different retinal cell populations are differentially labeled with labels that can be distinguished (e.g., using dyes or other labels having optical properties that can be distinguished from one another). This allows for the different cell populations to be sorted based on their different optical properties, either by specific absorbance or fluorescence spectra, or any distinctive and measurable combination thereof.

In some embodiments, retinal cells labeled in vitro or in vivo can be harvested and disaggregated from the retinal tissue, and then sorted by flow cytometry based on their distinctive optical properties, for example, by using fluorescence-activated cell sorting (FACS).

In some embodiments, the potential to sort labeled retinal cells may be confirmed in vivo, thus confirming the presence of dyes at the appropriate locations prior to harvesting the retinas. In some embodiments, an in vivo confirmatory analysis can be executed using scanning laser ophthalmoscopy (SLO).

### Vectors and Promoters for Transducing Retinal Cell Populations

In some embodiments, one or more recombinant vectors may be used to transduce one or more retinal cell populations. In some embodiments, one or more retinal cell populations are transduced with a gene that encodes a label (e.g., a fluorescent protein or protein fragment or other detectable protein or protein that generates a detectable signal). In some embodiments, one or more retinal cell populations are transduced with a test gene, vector, aptamer or protein (e.g., via protein transfection, for example using a liposomal molecule). Methods of administering either labels or test reagents may include injection (e.g., via intravitreal, subretinal, or sub-inner-lining-membrane (subILM) injection) or by topical application to the eye or via any other route of delivery to the eye. In some embodiments, a label or test reagent is administered systemically.

In some embodiments, one or more of the vectors utilized can be an adeno-assisted-virus (AAV), for example a recombinant AAV (rAAV). In some embodiments, one or more cell-type-specific promoters are utilized to drive expression of one or more transgenes that are delivered to targeted cell populations.

In some embodiments, a cell-type-specific promoter is human rhodopsin kinase promoter (hGRK1). Non-limiting examples of hGRK1 promoter can be found in Beltran et al., 2010, Gene Ther. 17:1162, Zolotukhin et al., 2005, Hum Gene Ther. 16:551, and Jacobson et al., Mol Ther. 13:1074. In some embodiments, a cell-type-specific promoter is a Pleiades Mini-promoter (for example Ple155). In some embodiments, such a cell-type-specific promoter is glial fibrillary acidic protein promoter. Other non-limiting examples of promoters that can be used as retinal cell-type-specific promoters include red opsin promoter “PR2.1” (M and L cones) chimeric ‘IRBPe-GNAT2’ promoter (all cones), IRBP promoter (rod targeted), Grm6-SV40 enhancer/promoter (bipolar cells), Thy1 (RGCs), other Pleiades promoters, rod opsin promoter (rods), cone arrestin promoters (all cones) VMD2 or Bestrophin promoter (RPE). In some embodiments, a PR2.1 promoter is used to target M and L cones. In some embodiments, a IRBPe-GNAT2 promoter is used to target all cones. In some embodiments, an IRBP promoter is used to target rods. In some embodiments, a Grm6-SV40 enhancer/promoter is used to target bipolar cells. In some embodiments, a Thy1 promoter is used to target RGCs. In some embodiments, a cone arrestin promoter is used to target cones. In some embodiments, a rod opsin promoter is used to target rods. In some embodiments, a VMD2 promoter (also known as Bestrophin promoter) is used to target RPE cells.

In some embodiments, a PR2.1 promoter is used to target M and L cones. In some embodiments, a IRBPe-GNAT2 promoter is used to target all cones. In some embodiments, an IRBP promoter is used to target rods. In some embodiments, a Grm6-SV40 enhancer/promoter is used to target bipolar cells. In some embodiments, a Thy1 promoter is used to target RGCs. In some embodiments, a cone arrestin promoter is used to target cones. In some embodiments, a rod opsin promoter is used to target rods. In some embodiments, a VMD2 or Bestrophin promoter is used to target RPE cells.

Several promoters are publically available or described. For example, Ple155 promoter is available through Addgene plasmid repository (Addgene plasmid #29011, https://www.addgene.org/29011/) and is described in scalabrino et al. (Hum Mol Genet. 2015, 24(21):6229-39). A Thy1 promoter construct is also available through Addgene plasmid repository (Addgene plasmid #20736, https://www.addgene.org/20736/). A GRM6 promoter construct is also available through Addgene plasmid repository (Addgene plasmid #66391, https://www.addgene.org/66391/). Guziewicz et al. (PLoS One. 2013 Oct. 15; 8(10):e75666) and Esumi et al (J Biol Chem. 2004, 279(18):19064-73) provide examples of the use of VMD2 promoter. Dyka et al. (Adv Exp Med Biol. 2014; 801: 695-701) describes cone specific promoters for use in gene therapy, including IRBP and IRBPe-GNAT2 promoter. The use of PR2.1 promoter has been demonstrated in Komáromy et al. (Gene Ther. 2008 July; 15(14):1049-55) and its characterization in Karim et al. (Tree Physiol. 2015 October; 35(10):1129-39). Aartsen et al. (PLoS One, 5(8):e12387) describes the use of GFAP promoter to drive GFP expression in Muller glial cells. Other examples of Muller glia specific promoters are RLBP1 and GLAST (Vazquez-Chona, Invest Ophthalmol Vis Sci. 2009, 50(8):3996-4003; Regan et al., Journal of Neuroscience, 2007, 27(25): 6607-6619).

It is to be understood that a promoter, as used methods disclosed herein may be as described in any of the references cited herein, or a fragment thereof that retains promoter activity.

In some embodiments, a non-specific promoter can be used to drive expression of a transgene in two or more different cell types. A non-specific promoter may be a constitutive promoter or an inducible promoter. For example, constitutive promoters of different strengths can be used. In some embodiments, more than one constitutive promoters are used. Such promoters may be viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter (e.g., chicken β-actin promoter) and human elongation factor-1α (EF-1α) promoter. Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Synthetic promoters are also contemplated herein. A synthetic promoter may comprise, for example, regions of known promoters, regulatory elements, transcription factor binding sites, enhancer elements, repressor elements, and the like.

In some embodiments, methods described in this application may be used in conjunction with the administration of one or more test vectors. In some embodiments, the test vectors are potential gene therapy vectors. Such vectors could be either rationally designed vectors to be tested singly or in small groups. In some embodiments, a large library of test vectors can be screened. Whether rationally designed or by library screening, the test vectors can be administered in vivo to a retina (e.g., in a non-human primate) that was either previously rendered wholly or partly sortable or that is subsequently rendered sortable. In some embodiments, the test vectors are either characterized by a specific and identifiable intrinsic genotype, or alternately they are encoded with a genetically engineered marker sequence (e.g., a genetic barcode), such that the genotype or genetic barcode can be recovered and/or identified subsequently from the sorted cells (e.g., by sequencing, hybridization, or other suitable technique).

In some embodiments, methods and compositions described in this application are useful to test the specificity and/or expression properties of one or more vectors in different retinal cell populations that can be determined subsequent to an in vitro, post-harvest, sorting of labeled and disaggregated retinal cells. In some embodiments, vector and/or nucleic acid libraries can be screened for on and off target delivery to a range of cell populations in the retina using a single, or a small number of, non-human primates.

In some embodiments, methods for sorting specific retinal cell populations described in this application can be used in conjunction with the administration of one or more model gene therapy vectors containing one or more test promoters, either rationally selected or as part of a library, and coupled with a reporter gene, for example, administered in vivo. Methods provided herein can be used to interrogate transduction profiles of existing vectors as well as the activity of regulatory elements (e.g., promoters) used for driving transgene expression. Methods provided herein also can be used as a screening method for identifying novel viral vectors, e.g., with capsid libraries, best suited for targeting individual retinal cell populations in a clinically relevant species.

Vectors and nucleic acids that are used for labeling or that are test reagents may be delivered to the retina by different methods. For example, vectors and/or nucleic acids (e.g., in liposomes) can be injected either subretinally, intravitreally, in the LGN, in sub-inner-limit-membrane, or topically administered, or delivered by any other suitable route.

### Validation of Retinal Cell Labeling and Transduction of a Gene

In some embodiments, the efficacy, specificity, and/or sequence identity of different test reagents (e.g., promoters or vectors) or labels in different retinal cells can be interrogated in the subsequently sorted cell populations (e.g., by assaying RNA and/or protein expression levels in different retinal cell types using any suitable assay technique).

In some embodiments, novel gene delivery vectors are engineered to contain unique nucleotide sequences, or barcodes, and the selective capture of a tissue or cell type of interest followed by next generation sequencing of recovered nucleic acids are used to generate a frequency distribution. In some embodiments, techniques such as RNA sequencing (also known as RNA-Seq or whole transcriptome shotgun sequencing) are used to assess alternative gene spliced transcripts, post-transcriptional modifications, gene fusion, mutations/SNPs and changes in gene expression over time, or differences in gene expression in different groups or treatments. In some embodiments, RNA is isolated and purified from cells, then a sequencing library is generated, which is then subsequently sequenced. In some embodiments, RNA-Seq is used to measure alternative gene spliced transcripts, post-transcriptional modifications, gene fusion, mutations/SNPs and changes in gene expression over time, or differences in gene expression in different groups or treatments. In some embodiments, cap analysis gene expression (CAGE-seq) is used to map the frequency and exact location of transcriptional start sites of different genes. In some embodiments, chromatin immunoprecipitation (ChIP)-sequencing (also known as ChIP-Seq) is used to assess binding sites of DNA-associated proteins, or to map global binding sites for any protein of interest. In some embodiments, such methods are used to assess the differences in the characteristics of RNA and DNA-binding proteins between different cell types (e.g., PR and RGCs). In some embodiments, different promoters can be evaluated based on their ability to regulate therapeutic transgene expression in a manner that delivers the transgene expression product to the targeted cell(s) of interest while minimizing the impact of off-target expression.

In some embodiments, targeting of a gene therapy delivery vector can be assessed by measuring the expression of genes in specific cell populations. Measuring expression of certain genes may be used to validate differential labeling of retinal cell types. For example, it may be tracked how well a label coincides with the expression of a gene that is specific to that cell type. Expression of such genes can be assessed using techniques such as quantitative real-time PCR. Non-limiting examples of retinal cell-specific genes include rhodopsin (RHO, rod specific), guanine nucleotide binding protein (G protein) subunit alpha transducing activity polypeptide 2 (GNAT2; cone specific), glutamate receptor metabotropic receptor 6 (GRM6; ON bipolar cell specific), glutamate-ammonia ligase/glutamine synthetase (GLUL; Muller cell specific), and Thy-1 cell surface antigen (THY1; retinal ganglion cell-specific). Non-limiting examples of cone specific genes included different cone opsins, M/L opsin, opsin1 (cone pigments), medium, or long-wave-sensitive (OPN1MW or OPN1LW) and S opsin, opsin1 (cone pigments), and short-wave-sensitive (OPN1SW). Non-limiting examples of RGC expressed genes include BRN3A, POU class4 homeobox (POU4F1), melanopsin and Opsin 4 (OPN4). Retinal pigment epithelium specific protein 65 kDa (RPE65) is a retinal pigment epithelium-specific gene.

In some embodiments, methods for sorting specific retinal cell populations described in this application can be used in conjunction with the administration of one or more functional gene therapy vectors, for example, administered in vivo. In some embodiments, the subsequent measurement of transgene expression in the sorted cell populations can be useful to identify gene therapy vectors that target one or more cell types of interest. In addition, the impact of transgene expression to cell or tissue function can be subsequently measured or evaluated in the sorted cell populations. The ability to measure the impact of the functional gene therapy vector can be used in the development of treatments or diagnoses for retinal specific diseases.

In some embodiments, the ability to measure the impact of functional gene therapy vector(s) using methods provided in this application can be used to facilitate pharmacological and toxicological studies that can be useful for designing and implementing gene therapy vector clinical trials in humans.

In some embodiments, selective labeling of bipolar cells is achieved via intravitreal or SubILM injection of a rAAV vector containing a bipolar specific promoter such as “Ple155” and a reporter gene. Similarly test reagents can also be delivered via intravitreal or SubILM injection of a rAAV vector containing a bipolar specific promoter such as “Ple155” and a reporter gene.

In some embodiments, RPE, astrocytes, microglia and horizontal cells can be targeted with AAV containing cell specific promoters. Selective labeling of Muller glia can be achieved following intravitreal injection of vital dyes such as Brilliant Blue G, Mitotracker Orange, Mitotracker Green, Celltracker Orange, Celltracker Green or monochlorobimane. Alternatively, intravitreal injection of existing AAV vectors containing Muller glia specific promoters such as RLBP1, GLAST or GFAP may be employed.

In some embodiments, NHPs with selectively labeled retinal cell populations can be used to evaluate and select vectors and other molecules which target retinal cells of interest. Vectors can include viral vectors such as AAV, Adenovirus, Lentivirus, or other viral vectors. Other molecules can include nucleic acid (e.g., DNA) aptamers.

In some embodiments, an AAV capsid library containing many (e.g., tens, hundreds, thousands or millions of) different capsids which contain genomes either matched to the capsid (for example that code for that capsid), or a barcoded sequence that identifies the capsid can be delivered to the eye (e.g., intravitreally, subretinally or in a particular region of the brain such as the LGN). Thereafter, the NHP retina is dissociated and the desired/labeled cell population can be sorted via FACS. In some embodiments, analysis of DNA within the sortable cell population can be used to identify which AAV capsid sequence is most enriched in retinal cells of interest, thereby identifying, for example, one or more capsid sequences that can be useful to target these retinal cells.

As disclosed herein, a method of preparing a non-human primate retina comprising one or more sortable retinal cell populations, in some embodiments, involves different rAAV particles used for example for two purposes. The first purpose is to label a particle cell type in a retina (e.g., labeling PRs with an rAAV particle comprising a gene that encodes a fluorescent protein such as EGFP). The other purpose is to test the activity and cell tropism of an rAAV particle for different retinal cells or to test the specificity or activity of test genetic constructs in different retinal cells. In some embodiments, an rAAV particle used to label a particular retinal cell type transduces cells of non-human primates and/or retinal cells. In some embodiments, AAV particles that transduce non-human primate retinal cells are of serotype 5, 7, 8 or 9. In some embodiments, AAV particles of serotype 5, 8 or 9 are used to label PRs, or retinal pigment epithelium (RPE) cells. In some embodiments, AAV9 particles are used to target cone PRs (Vandenberghe et al., PLoS One. 2013; 8(1): e53463). In some embodiments, a variant or hybrid AAV particle is used, e.g., AAV2, AAV5 or AAV8 particles in which surface exposed tyrosine (Y) and/or threonine (T) residues are changed to phenylalanine (F) and/or valine (V), respectively (Kaye et al., PLoS One. 2013, 8(4):e62097), or an AAV8 particle in which amino acids 587-595 in the VP1 capsid protein is mutated (US20150376240 A1, which is incorporated herein by reference in its entirety).

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. Replication proteins are expressed from the rep ORF, while capsid proteins are expressed from the cap ORF. Capsid proteins form the capsid of a virus particle. In some embodiments, capsids are empty. In some embodiments, recombinant AAV (rAAV) particles comprise a nucleic acid vector, which may comprise one or more of the following: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, a nucleic acid vector in a rAAV particle comprises one or more nucleic acid regions comprising a sequence that facilitates expression of the heterologous nucleic acid region (e.g., a promoter). In some embodiments, a nucleic acid vector in a rAAV particle comprises one or more nucleic acid regions comprising a sequence that facilitate integration of the heterologous nucleic acid region (optionally with the one or more nucleic acid regions comprising a sequence that facilitates expression) into the genome of the subject.

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Calif.; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci U S A. 1996 Nov. 26; 93(24):14082-7; and Curtis A. Machida. Methods in Molecular Medicine™. Viral Vectors for Gene Therapy Methods and Protocols. 10.1385/1-59259-304-6:201© Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Genebank reference numbers for sequences of AAV serotypes 1, 2, 3, 3B, 4, 5, 6, 7, 8, 9, 10, 11, 12, and 13 are listed in patent publication WO2012064960, which is incorporated herein by reference in its entirety. The rAAV particle or particle within an rAAV preparation may be of any AAV serotype, including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9).

Methods of producing rAAV particles and nucleic acid vectors are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into a recombinant cells such that the rAAV particle can be packaged and subsequently purified.

A gene of interest may be a gene that encodes a protein that is a detectable marker (e.g., fluorescent or bioluminescent protein) that can serve as a label, or a gene that includes a regulatory sequence and/or that encodes a protein that is being evaluated in a method described herein.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprises rep and/or cap genes that encode the Rep protein and/or Cap proteins for use in a method described herein. In some embodiments, the packaging is performed in vitro.

In some embodiments, a plasmid containing the gene of interest is combined with one or more helper plasmids, e.g., that contain a rep gene of a first serotype and a cap gene of the same serotype or a different serotype, and transfected into helper cells such that the rAAV particle is packaged.

In some embodiments, the one or more helper plasmids include a first helper plasmid comprising a rep gene and a cap gene, and a second helper plasmid comprising one or more of the following helper genes: E1a gene, E1b gene, E4 gene, Ea gene, and VA gene. For clarity, helper genes are genes that encode helper proteins E1a, E1b, E4, E2a, and VA.

Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Calif.; and Addgene, Cambridge, Mass.; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically

Controllable, and Two-Plasmid-Based Production of Adeno-associated Virus Vectors of Serotypes 1 to 6, Molecular Therapy,Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adeno-associated viral vector reference standards, Molecular Therapy, Vol. 16, 1185-1188). Plasmids that encode wild-type AAV coding regions for specific serotypes are also know and available. For example pSub201 is a plasmid that comprises the coding regions of the wild-type AAV2 genome (Samulski et al. (1987), J Virology, 6:3096-3101).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus and herpesvirus.

Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsC1 gradient, chromatography, or polyethylene glycol (PEG) precipitation.

### Labels

In some embodiments, a label to differentiate different retinal cells are proteins expressed within the cell, e.g., a fluorescent protein or protein fragment. In some embodiments, fluorescent protein is a blue fluorescent protein, a cyan fluorescent protein, a green fluorescent protein, a yellow fluorescent protein, an orange fluorescent protein, a red fluorescent protein, or functional peptides or polypeptides thereof.

In some embodiments, one or more retinal cell populations are labeled by injection of one or more different labels (e.g., dyes and/or genes that express detectable proteins). In some embodiments, a label is administered by injection to the lateral geniculate nucleus, resulting in retrograde migration of the label to retinal ganglion cell bodies. In some embodiments, a dye administered to the lateral geniculate nucleus is TRITC/Biotin-labeled Dextran (also known as MICRO-RUBY™).

In some embodiments, a label is administered by injection (e.g., via intravitreal, subretinal, or sub-inner-lining-membrane (subILM) injection) or topically or via any other suitable route. In some embodiments, the label that is administered is a vital dye. In some embodiments, the label that is administered is Brilliant Blue G, Mitotracker Orange, Mitotracker Green, Celltracker Orange, Celltracker Green and/or other suitable dye. In some embodiments, the label that is administered is monochlorobimane.

In some embodiments, a label is administered to a retina in vivo (e.g., in situ in a mammal, for example a non-human primate). However, in some embodiments, a label is introduced after harvest of the eye, but prior to disassociation of cells from the tissue, for example by bathing a portion of tissue in the labeling agent which then moves in a retrograde manner to label specific cells or tissues.

In some embodiments, methods described in this application can be used to identify and/or isolate regulatory elements (e.g., promoters) that are capable of driving gene expression in different retinal cell types (e.g., cell-type specifically). This technology can be used to interrogate transduction profiles of novel vectors and/or promoters as well as identify those that are suited for addressing various forms of retinal disease. For example, a library of constructs (e.g., viral constructs) containing different regulatory elements (e.g., promoters) can be introduced to the eye either before, after or during differential labeling of retinal cells. Thereafter, the different cell types of the retina can be identified based on the labels and an assessment of detecting the expression of the gene regulated by the promoters can be performed.

In some embodiments, one or both retinas of a single model animal may be used. In some embodiments, additional retinas may be used depending on the number of experiments and/or the statistical significance of the information that is being obtained.

In some embodiments, the cells to be labeled in the retina are PRs and RGCs. In some embodiments, PRs and RGCs are labeled in-life, whereas in some embodiments, PRs and RGCs are labeled ex-vivo. In some embodiments, cell labeling is followed by cell isolation. Cells can be isolated using dissociation methods, for example incubation in a sample of papain, or papain and DNAse under condition of constant agitation.

In some embodiments, for ex vivo labeling of cells, eyes are enucleated and the animal immediately euthanized. The anterior chamber and the vitreous are then removed and the resulting eyecup is immersed in oxygenated Ames media while the retina is isolated from the retinal pigment epithelium. In some embodiments, the retina is dissected in different parts, e.g., quadrants. In some embodiments, to retrogradely label RGCs, the eye is stabilized with the optic nerve end up and a small piece of tygon tubing is attached to the back of the eye cup, creating a well around the severed end of the optic nerve. A label, e.g., MICRO-RUBY™ is added to the well and allowed to incubate for a finite amount of time (e.g., 1, 2, 3, or 4 hours at room temperature, or longer periods of time at lower temperatures). In some embodiments, to label PRs ex vivo, dissociated cells from the retina are incubated with a label that is different from the one used to label RGCs (e.g., FITC conjugated peanut agglutinin).

In some embodiments, for in vivo or in-life labeling of cells, PRs are labeled by subretinally injecting a label, or viral vector or particle containing a GFP under the control of a promoter (e.g., hGRK1) into the subretinal space. In some embodiments, a few days after subretinal injection of the label for PRs (e.g., 1-40, 1-10, 5-20, 15-30 or 20-30 days), the LGN are injected bilaterally with a label for uptake by the RGC axonal projections, e.g., MICRO-RUBY™ to retrogradely label RGC cell bodies. In some embodiments, fundus imaging to confirm PR labeling is performed a few days (e.g., 2-10, or 3-6 days) before the LGN injections. In some embodiments, fundus imaging is performed a few days (e.g., 5-30, 10-25 or 18-24 days) after subretinal injections of the label for PRs. Thereafter, enucleation and subsequent tissue process, as described above for ex vivo labeling of cells, is performed 3-10 days (e.g., 3, 4, 5, 6, 7, 8, 9 or 10) days after LGN injections.

### EXAMPLES

### Example 1: Ex Vivo Labeling and Sorting of Retinal Cell Types

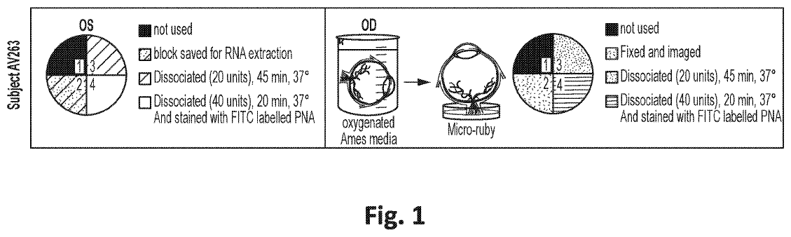

A non-limiting example of a method for labeling different retinal cell types ex vivo is illustrated in FIG. 1. A first enucleated eye (the left eye of a non-human primate) is dissected to remove the retina. Different quadrants of the retina are processed differently, including one quadrant that is blocked (e.g., with RPE into RNAlater) and saved for RNA extraction, one quadrant that is dissociated under a first set of conditions (dissociated 20 units for 45 min. at 37 C), and one quadrant that is dissociated under a second set of conditions and stained with FITC labelled PNA (dissociated 40 units for 20 min. at 37 C, and dissociated cells were incubated with 5 microg/mL PNA (1 mg/ml diluted 1:20) in 0.1 M PNS/5% FBS for 15 min. at RT, and 400 microL cell suspension was fixed for 20 min. in 4% PFA, spun, rinsed and plated for imaging, the remainder was used for cell sorting).

A second enucleated eye (the right eye of the non-human primate) was incubated for 2-3 hours in oxygenated Ames media, and dipped into a MICRO-RUBY™ solution (2.5 mg/mL MICRO-RUBY™ diluted in oxygenated Ames Media) to label photoreceptor cells via retrograde transport. The eye was stabilized with the optic nerve end up, a small piece of tygon tubing was attached to the back of the eye cup, creating a well around the severed end of the optic nerve. The retina is dissected and different quadrants are either fixed and imaged, dissociated under a first set of conditions, or dissociated under a second set of conditions and stained with FITC labeled PNA to label cone photoreceptors. A summary of the experimental design for ex vivo cell labeling is shown in FIG. 1.

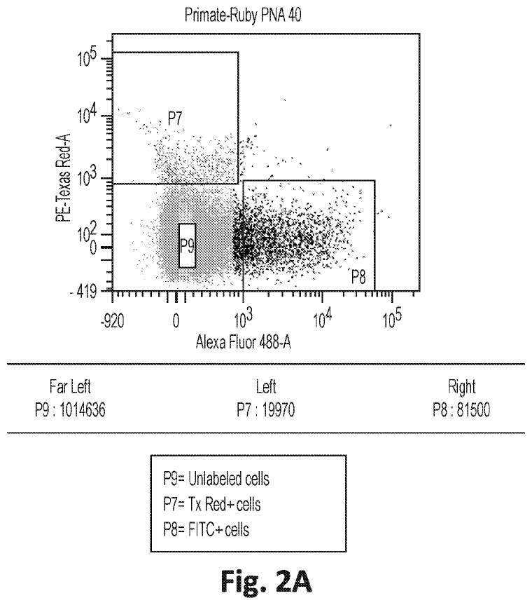

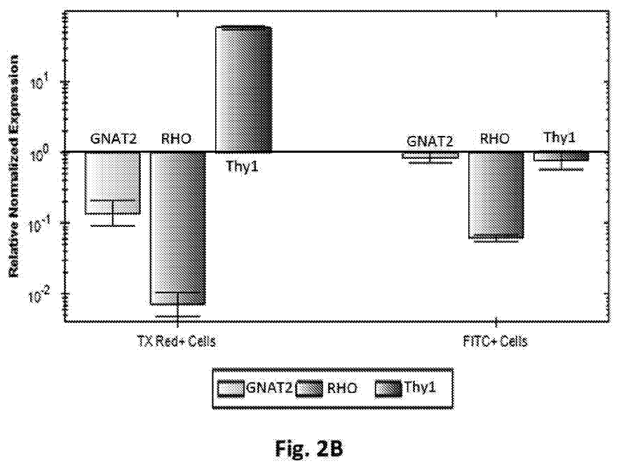

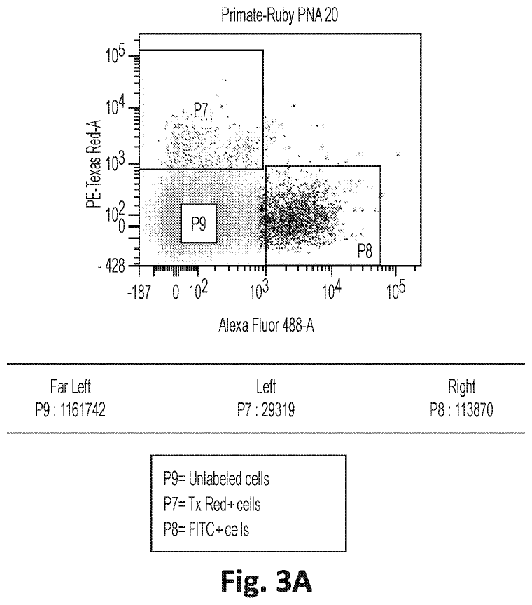

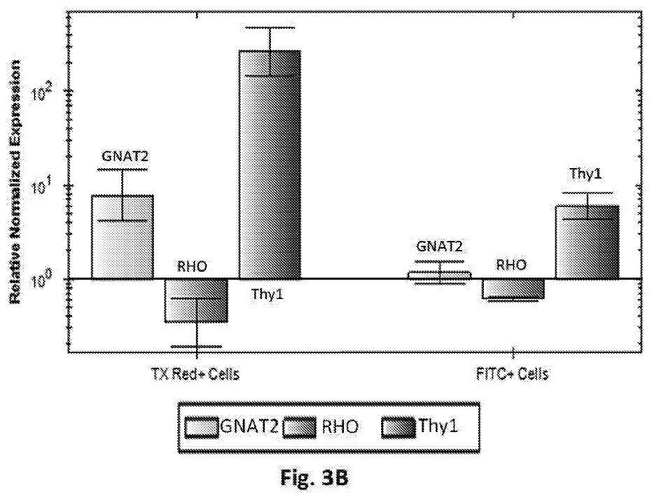

Cells from the right eye (4^{th} quadrant) were evaluated in two separate experiments illustrated in FIGS. 2 and 3. Initially, the differentially labeled cells were sorted via FACS as illustrated in FIGS. 2A and 3A. Each figure shows that the retinal tissue include unlabeled cells, cells labeled in Tx Red (MICRO-RUBY™ labeled cells), and FITC labeled cells, all of which can be separated via FACS sorting. The sorted cells were then assayed (via transcript analysis) to determine the relative expression levels of the following different genes GNAT2 (characteristic of and expressed at relatively higher levels in cone photoreceptor cells), RHO (characteristic of and expressed at relatively higher levels in rod photoreceptor cells), and THY1 (characteristic of and expressed at relatively higher levels in retinal ganglion cells). The expression levels in Tx Red labeled cells and FITC labeled cells are normalized relative to unlabeled cells and the results are shown in FIGS. 2B and 3B. The results show that the separation of cells via differential labeling targeted to retinal ganglion cells and photoreceptor cells respectively produces different cell populations that are characterized by different gene expression profiles that are consistent with the expected retinal cell types in the sorted populations.

### Example 2: In Vivo Labeling and Sorting of Retinal Cell Types

NHP were subretinally injected with AAV5 containing human rhodopsin kinase promoter (hGRK1)—driving GFP. GFP expression was validated four weeks post-injection using SLO. The lateral geniculate nuclei were then injected bilaterally with TRITC/Biotin-labeled Dextran (MICRO-RUBY™) to retrogradely label RGCs. Labeling of each cell type can be done alone or in combination as long as reporters of different wavelengths are chosen. Labeling is confirmed with in-life imaging of NHP retinas using scanning laser ophthalmoscopy (SLO).

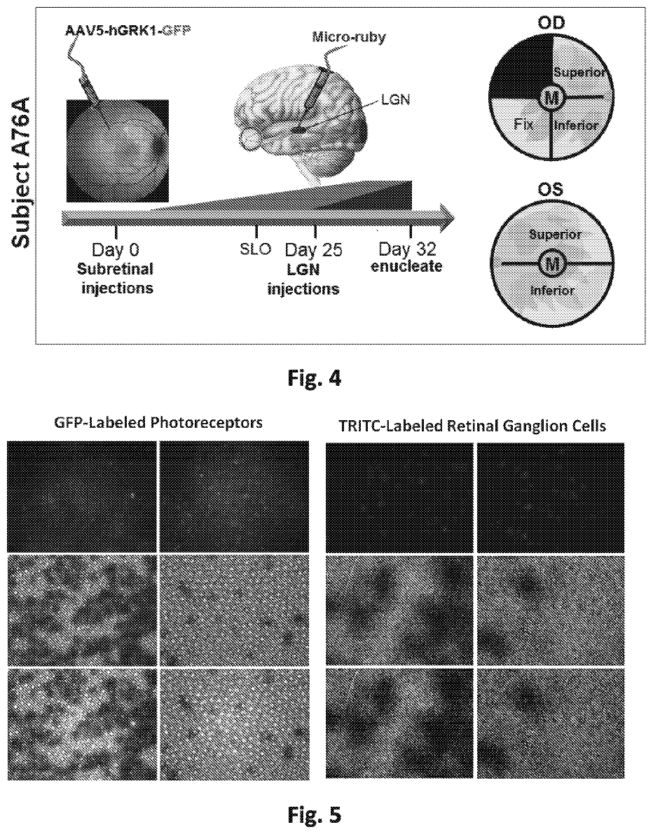

Eyes were enucleated and samples corresponding to macular, superior and inferior retina were dissociated using conditions optimized for NHP retina as illustrated in FIG. 4. To isolate the fovea, a 4 mm punch was made around the macula for both eyes. The resulting retinal tissue included GFP-labeled photoreceptors (PRs) and TRITC-labeled retinal ganglion cells (RGCs) when verified by retinal whole mount. Briefly, after fovea/macula were punched out, the remaining left retina was divided into superior and inferior hemispheres. The right retina minus the macula was divided into four equal quadrants as shown in FIG. 4. Quadrant 1 (temporal superior retina) was stored away. Quadrant 2 (temporal inferior retina) was fixed in PFA and mounted for imaging. Quadrants 3 and 4 (nasal inferior and nasal superior retina, respectively) were combined with the inferior and superior regions from the left retina, respectively to constitute the “superior” and “inferior” samples. The macula/foveal punches from both eyes were combined to constitute the “macula/fovea” sample. Samples were dissociated and analyzed by FACS.

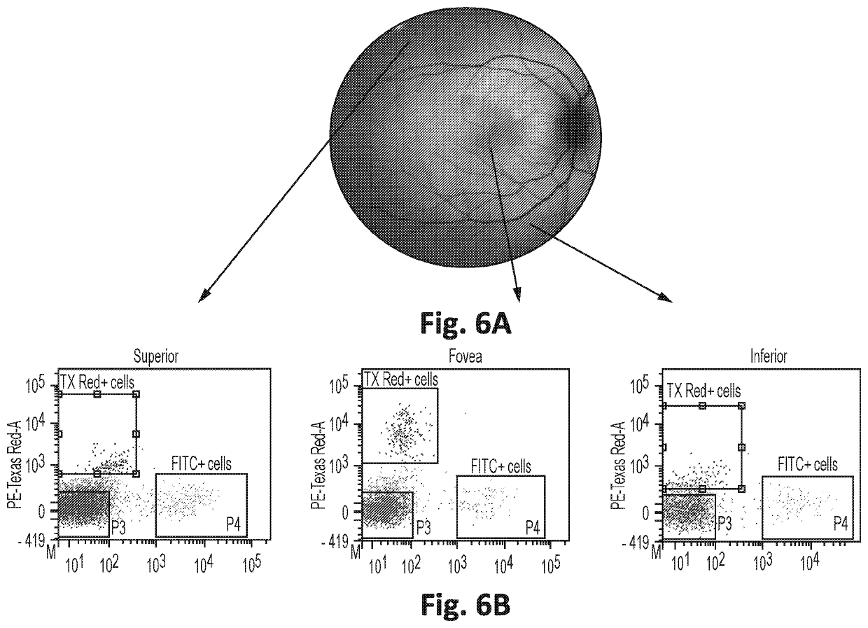

FIG. 5 illustrates GFP+and MICRO-RUBY™+labeling of putative PRs and RGCs respectively.

The dissociated cells were FACS sorted. Relative expression of retinal cell specific genes in the GFP+and MICRO-RUBY™+populations from the respective geographic locations were compared to unlabeled cells by qRT-PCR.

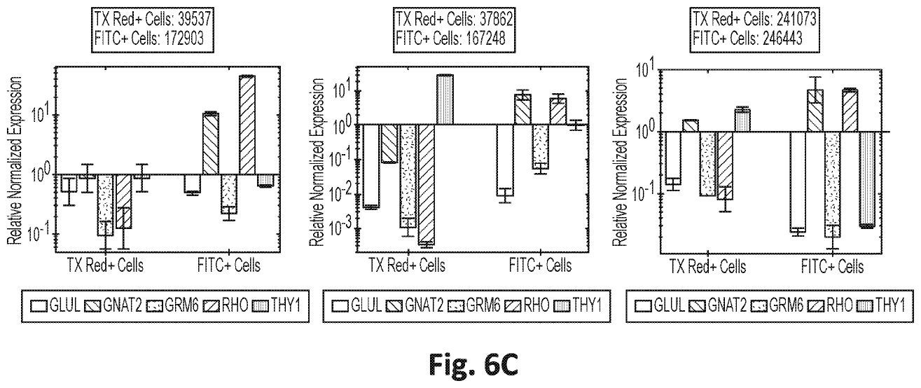

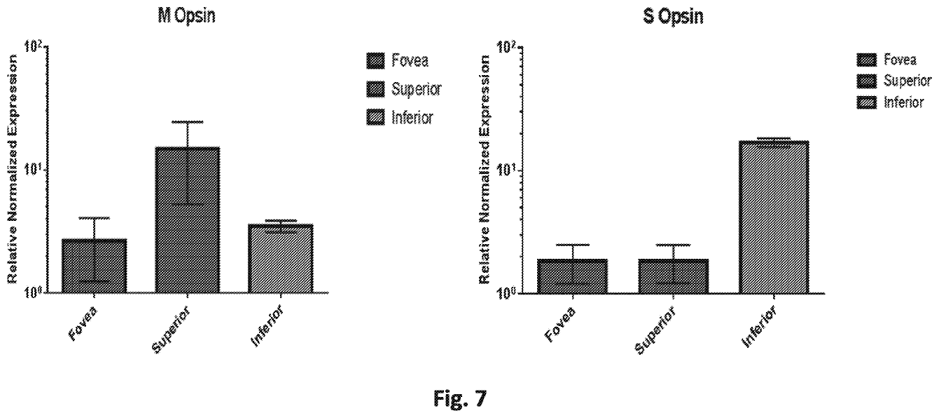

FIG. 6A shows the different retinal regions (superior retina, fovea, and inferior retina) from which labeled cells were obtained. FIG. 6B shows cell sorting (using FACS) of the differentially labeled cells from the superior retina, fovea, and inferior retina, in the left, center, and right panels respectively. FIG. 6C shows different gene expression profiles of the different retinal cell populations sorted as illustrated in FIG. 6B for the superior retina, fovea, and inferior retina, in the left, center, and right panels respectively. The gene expression of the following genes was evaluated: GLUL (characteristic of and expressed at relatively higher levels in Muller glial cells), GNAT2 (characteristic of and expressed at relatively higher levels in cone photoreceptor cells), GRM6 (characteristic of and expressed at relatively higher levels in On bipolar cells), RHO (characteristic of and expressed at relatively higher levels in rod photoreceptor cells), and THY1 (characteristic of and expressed at relatively higher levels in retinal ganglion cells). These gene expression profiles in GFP+and MICRO-RUBY™+cells indicated isolation of pure PR and RGC populations, respectively. Furthermore, variation in M/L cone, S cone, and rod specific gene expression from sorted PRs correlated with the relative abundance of these cell types in respective retinal regions. FIG. 7 illustrates a non-limiting example of M opsin and S opsin expression in fovea, superior retinal cells, and inferior retinal cells that were sorted based on differential labeling.

These results confirm that different retinal cell types can be differentially labeled in vivo, and the differentially labeled cells can be dissociated and sorted. The sorted cells correspond to different cell types (e.g., based on gene expression profiles). This procedure can be used in conjunction with the delivery of one or more agents of interest to the eye and/or retina of a non-human primate to determine the cell type specificity and/or targeting effectiveness of the one or more agents. These techniques be useful to identify and develop therapeutic agents having desired cell targeting properties.

Materials and Methods applicable to Example 1 and Example 2

### AAV Vector Preparation and Subretinal Injections

An AAV vector comprising the 292 base pair human rhodopsin kinase promoter (hGRK1) driving GFP packaged in AAV serotype 5 was used. Virus stock at a titer of 8×10¹³ vector genomes per milliliter (vg/ml) was diluted in the same buffer used for AAV storage (balanced salt solution with 0.014% Tween-20) to the delivered dose concentration of 1×10¹² vg/ml approximately 1 h prior to subretinal injections. Vector dosing solutions were allowed to warm to room temperature to avoid off-gassing during injection. Subretinal injections were performed as described in Boye et al., 2012, Hum Gene Ther, 23:1101.

### MICRO-RUBY™ Preparation and LGN Injections

Tetramethylrhodamine and biotinylated dextran 3000 MW, lysine fixable was reconstituted to 10% in sterile saline. 25 days post-subretinal injection and 5 days after fundus imaging, the LGN were injected bilaterally with MICRO-RUBY™ to retrogradely label RGCs.

### In Vivo Imaging

In-life GFP expression was observed 20 days post-injection using a Topcon TRC 50EX fundus camera equipped with a Fundus Photo adapter.

### Enucleation

At the time of sacrifice (32 days post-subretinal injections for animal in which cells were labeled in-life), animals were deeply anesthetized and eyes were enucleated. The animals were euthanized immediately thereafter. The anterior chamber and the vitreous were removed and the resulting eyecup was immersed in oxygenated Ames media while the retina was isolated from the retinal pigment epithelia.

### Retinal Dissociation and FACS

Retinal samples were dissociated with papain (Worthington Biochemical Corporation, NJ, Catalog #3150) according to the manufacturer's protocol. Dissected retina samples were placed in a solution of papain and DNase and equilibrated with 95% O₂:5% CO₂ at 37° C. for 20-45 minutes with constant agitation. Dissociated cells were collected by centrifugation, resuspended and stained using standard staining procedures.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of preparing a non-human primate retina comprising one or more sortable retinal cell populations, the method comprising labeling a first retinal cell type in the non-human primate retina with a first label.

2. The method of claim 1, wherein the one or more sortable retinal cell populations is selected from the group consisting of: photoreceptors (PR), retinal ganglion cells (RGC), biopolar cells, retinal pigment epithelium (RPE), astrocytes, horizontal cells, amacrine cells, microglia, and Muller glia.

3. The method of any of the preceding claims, wherein one or more retinal cell populations is rendered sortable by labeling with a dye.

4. The method of any of the preceding claims, wherein one or more additional cell populations is differentially labeled with one or more additional dyes.

5. The method of any of the preceding claims, wherein the one or more sortable retinal cell populations are sorted using Fluorescence-activated cell sorting (FACS).

6. The method of any of the preceding claims, wherein the one or more sortable retinal cell populations are labeled in vivo, and optionally wherein confirmation of differential in vivo labeling of different retinal cell types is confirmed in vivo.

7. The method of any of the preceding claims, wherein the in vivo confirmatory method is scanning laser ophthalmoscopy (SLO) or fundoscopy.

8. The method of any of the preceding claims, wherein one or more recombinant vectors are used to transduce the one or more sortable retinal cell populations.

9. The method of claim 8, wherein the vector is an adeno associated virus (AAV).

10. The method of any of the preceding claims, where one or more cell-type-specific promoters are utilized to drive expression of one or more transgenes in target retinal cell populations.

11. The method of claim 10, wherein the one or more cell-type-specific promoters are selected from the group consisting of: human rhodopsin kinase promoter (hGRK1), a Pleiades Mini-promoter (Ple155), a glial fibrillary acidic protein (GFAP) promoter, a red opsin promoter (PR2.1), a chimeric IRBPe-GNAT2 promoter, an IRBP promoter, a Grm6-SV40 enhancer/promoter, Thy1, VMD2 and Bestrophin promoter.

12. The method of any of the preceding claims, wherein the one or more retinal cell populations is labeled by injection of a dye.

13. The method of claim 12, wherein the injection is administered to the lateral geniculate nucleus, and wherein the dye is TRITC/Biotin-labeled Dextran (MICRO-RUBY™).

14. The method of claim 12 or 13, wherein the injection is administered intravitreally.

15. The method of any of claims 12-14, wherein the injected dye is selected from the group consisting of: Brilliant Blue G, Mitotracker Orange, Mitotracker Green, Celltracker Orange, Celltracker Green, and monochlorobimane.

16. The method of any of the preceding claims, wherein the labeling agent is administered by intravitreal injection or by sub-inner-limit-membrane injection (subILM).

17. The method of any of the preceding claims, wherein the labeling agent is introduced by bathing a portion of tissue in the labeling agent which then moves in a retrograde manner to label specific cells or tissues.

18. The method of any of the preceding claims, further comprising administering one or more test vectors to the retina in vivo.

19. The method of any of the preceding claims, further comprising administering one or more vectors containing one or more test promoters with a reporter gene to the retina in vivo.

20. The method of any of the preceding claims, further comprising administering one or more functional gene therapy vectors to the retina in vivo.

21. The method of any prior claim, further comprising determining an expression level or activity level of one or more labels, transgenes, promoters, or other agents in one or more sortable cell types.

22. The method of claim 21, further comprising comparing the expression level or activity level to a reference level.

23. The method of claim 22, further comprising determining whether one or more labels, transgenes, promoters, or other agents are more highly expressed or more active in a first retinal cell type relative to a second retinal cell type.
